(19) United States
(12) Patent Application Publication
(10) Pub. No.: US 2023/0096313 A1
(43) Pub. Date: Mar. 30, 2023

(54) METHOD OF BIOMARKER DETECTION IN COMPANION ANIMALS

(71) Applicant: Uni Pharma Co., LTD., Taipei City (TW)

(72) Inventor: Yu-Shan Wang, Hsin-Chu (TW)

(21) Appl. No.: 17/945,709

(22) Filed: Sep. 15, 2022

Related U.S. Application Data
(60) Provisional application No. 63/249,329, filed on Sep. 28, 2021.

(51) Int. Cl. G01N 33/86; G01N 1/40; G01N 33/574; G01N 33/543

(52) U.S. Cl. CPC G01N 33/86; G01N 1/4077; G01N 33/57484; G01N 33/54386; G01N 2001/4083; G01N 2470/04

(57) ABSTRACT

Two methods for quantifying fibrin/fibrinogen degradation products (“FDP”) in a blood sample. The first method features obtaining a blood sample from a non-human animal, centrifuging the blood sample to obtain a first supernatant and collecting the first supernatant, centrifuging the first supernatant to obtain a second supernatant and collecting the second supernatant, diluting the second supernatant, contacting the diluted second supernatant with a reagent that contains antibodies specifically binding to FDP, and detecting an amount of antibodies specifically bound to FDP, thereby quantifying FDP in the blood sample. The second method requires subjecting the diluted second supernatant to a quantitative immunoassay that specifically detects FDP to quantify the amount of FDP in the non-human blood sample.

### CROSS-REFERENCE TO RELATED APPLICATIONS

The present application claims the benefit of U.S. Provisional Patent Application Ser. No. 63/249,329 filed on Sep. 28, 2021, the content of which is hereby incorporated by reference in its entirety.

### BACKGROUND

Companion animals, like humans, are living longer due to advances in medicine and improvements in preventive care and nutrition. Pets having longer lifespans are more likely to develop diseases of aging, cancer in particular. An estimated 6 million dogs and nearly 6 million cats are diagnosed with cancer each year.

There are nearly 100 types of animal cancer. Cancer in pets can be found in the skin, bones, breast, head and neck, lymph system, abdomen, and testicles. Leukemia is the most common type of cancer in cats and lymphoma and mammary gland cancer are the most common type of cancer in dogs.

Dogs are affected by more forms of cancer compared to other companion animals According to The US Veterinary Cancer Society, cancer is the leading cause of death in 47% of dogs, especially dogs over age ten.

Many biomarkers present in human serum are known to be associated with cancer, e.g., carcinoembryonic antigen and fibrin/fibrinogen degradation products. Certain biomarkers have been determined to be predictive of the progression of human cancer. However, studies on non-human animal cancer remain sporadic.

The need exists to develop methods to detect biomarkers associated with animal cancers so that appropriate treatment can be administered at an earlier stage in the cancer, leading to improved survival.

### SUMMARY

To meet this need, a method for quantifying fibrin/fibrinogen degradation products (“FDP”) in a blood sample is provided. The method is carried out by obtaining a blood sample from a non-human animal, centrifuging the blood sample to obtain a first supernatant and collecting the supernatant, centrifuging the first supernatant to obtain a second supernatant and collecting the second supernatant, diluting the second supernatant, contacting the diluted second supernatant with a reagent that contains antibodies specifically binding to FDP, removing antibodies not bound to FDP, and detecting an amount of antibodies specifically bound to FDP, thereby quantifying FDP in the blood sample.

Also provided is a second method for quantifying FDP in a blood sample. This method is accomplished by obtaining a blood sample from a non-human animal, centrifuging the blood sample to obtain a first supernatant and collecting the supernatant, centrifuging the first supernatant to obtain a second supernatant and collecting the second supernatant, diluting the second supernatant, and subjecting the diluted second supernatant to a quantitative immunoassay that specifically detects FDP to quantify the amount of FDP in the blood sample.

The details of several embodiments of the present invention are set forth in the description below. Other features, objects, and advantages of the invention will be apparent from the description and the drawings, and also from the appended claims.

### BRIEF DESCRIPTION OF THE DRAWINGS

The description below refers to the accompanying drawings, of which:

FIG. 1A shows a plot of expression levels of DR-70 antigen in control dogs and tumor-bearing dogs, and

FIG. 1B shows a receiver operator characteristic curve calculated from the data presented in FIG. 1A and constructed from a range of cutoff values.

### DETAILED DESCRIPTION

As summarized above, a first method is disclosed for quantifying FDP in a blood sample. The method features a step of centrifuging a non-human blood sample to obtain a first supernatant.

Preferably, the non-human blood sample is from a mammal, e.g., a dog, a cat, a rabbit, a guinea pig, a ferret, or a pig. Blood can be obtained by any method known in the art.

In a particular first method, anti-coagulated blood can be used. For example, fresh blood can be treated with ethylenediaminetetraacetic acid or sodium citrate to prevent coagulation. Anti-coagulation of the non-human blood sample is not required to carry out the claimed invention, as clotted whole blood can also be used.

The non-human blood sample is centrifuged to remove blood cells so that a first supernatant is obtained. The centrifugation can be carried out at 10×g to 2,000×g (e.g., 10×g, 50×g, 100×g, 250×g, 500×g, 1000×g, 1250×g, 1500×g, and 2000×g) for a period of 30 s to 30 min. (e.g., 30 s, 1 min., 5 min., 10 min., 15 min., 20 min., 25 min., and 30 min.).

After collecting the first supernatant, e.g., by pipetting it from atop the blood cell pellet, the first supernatant is centrifuged again to obtain a second supernatant. This second centrifugation step is carried out at 1,000×g to 50,000×g (e.g., 1,000×g, 2,500×g, 5,000×g, 10,000×g, 11,000×g, 12,000×g, 13,000×g, 14,000×g, 15,000×g, 16,000×g, 17,000×g, 18,000×g, 19,000×g, 20,000×g, 25,000×g, 30,000×g, 35,000×g, 40,000×g, 45,000×g, and 50,000×g) for 30 s to 30 min. (e.g., 30 s, 1 min., 5 min., 10 min., 15 min., 20 min., 25 min., and 30 min.).

After collecting the second supernatant, it is prepared for immunodetection by diluting it with an appropriate diluent. For example, phosphate buffered saline (“PBS”) can be used to dilute the second supernatant.

The second supernatant can be diluted 1:5 to 1:10,000, preferably 1:50 to 1:5,000, and more preferably 1:100 to 1:1,000. Acceptable dilutions can be, e.g., 1:5, 1:25, 1:50, 1:100, 1:200, 1:300, 1:400, 1:500, 1:750, 1:1,000, 1:1,500, 1:2,000, 1:2,500, 1:3,000, 1:3,500, 1:4,000, 1:4,500, 1:5,000, 1:7,500, and 1:10,000.

The diluted second supernatant is then placed in contact with a reagent that contains antibodies specifically binding to FDP.

It is within the skill of an ordinary artisan to develop antibodies specifically binding to FDP. For example, FDP isolated from an animal of interest, e.g., a dog, can be used to inoculate a rabbit to obtain a polyclonal serum that contains antibodies specifically binding to FDP. Mice can be inoculated with FDP and splenocytes isolated from positive responding animals to obtain monoclonal antibodies against FDP. In a particular example, certain antibodies that bind to human FDP cross-react with FDP from other species. As such, anti-human FDP polyclonal or monoclonal antibodies can be used as the reagent.

The above method requires that the diluted second supernatant be placed in contact with the reagent. This can be accomplished, for example, by adsorbing the diluted second supernatant to a plate surface and exposing the plate surface to the reagent that contains anti-FDP antibodies. In another example, the diluted second supernatant is mixed in a tube with the reagent.

After allowing for sufficient time for the anti-FDP antibodies in the reagent to bind to FDP in the diluted second supernatant, unbound antibodies are removed. For example, if the diluted second supernatant is adsorbed to a plate, the unbound anti-FDP antibodies can be removed by washing with an appropriate detergent solution, e.g., PBS/polysorbate-20. Further, if the diluted second supernatant is mixed in a tube with the reagent, unbound antibodies can be separated and removed from bound antibodies by adding a second antibody that also binds to FDP and capturing the second antibody.

As mentioned, supra, the method includes a step of detecting the amount of antibody specifically bound to FDP after the contacting step. The anti-FDP antibody in the reagent can be fluorescently or enzymatically labelled and the amount of anti-FDP antibodies bound to FDP can be measured, e.g., by fluorescence intensity, if using a fluorescently labelled antibody, or by colorimetric means, if using an enzymatically labelled antibody. Any method known in the art for detecting the specifically bound anti-FDP antibodies can be used.

The amount of FDP in the second supernatant can be quantified in a number of ways. In a particular example, the amount of anti-FDP antibodies bound to a standard of purified FDP at different dilutions is compared to the amount detected in the second supernatant.

The above method can be performed on blood samples from several animals of the same species for comparative purposes. For example, the method can be used to determine FDP levels in blood samples from dogs that are free of cancer to establish a baseline value. The same method can be used to determine FDP levels in blood samples from dogs having cancer to learn whether these levels are different from those in cancer-free animals.

Mentioned in the summary section is a second method for quantifying FDP in a blood sample. The second method shares certain steps with the first method set out in detail above. The second method, like the first method, requires the steps of (i) obtaining a blood sample from a non-human animal, (ii) centrifuging the blood sample to obtain a first supernatant (iii) collecting the first supernatant, (iv) centrifuging the first supernatant to obtain a second supernatant, (v) collecting the second supernatant, and (vi) diluting the second supernatant. The second method features a step of subjecting the diluted second supernatant to a quantitative immunoassay that specifically detects FDP in order to quantify the amount of FDP in the blood sample.

The second method, like the first method described above, does not require anti-coagulation of the non-human blood sample. Clotted whole blood can be used.

The centrifuging steps and dilutions in the second method are performed in the same manner as the first method. To reiterate, the non-human blood sample is centrifuged at 10×g to 2,000×g for 30 s to 30 min. to obtain the first supernatant, the first supernatant is centrifuged at 1,000×g to 50,000×g for 30 s to 30 min. to obtain the second supernatant, and the second supernatant is diluted 1:5 to 1:10,000 for use in the quantitative immunoassay.

The quantitative immunoassay can be, but is not limited to, an enzyme-linked immunosorbent assay (“ELISA”), an enzyme multiplied immunoassay technique, a quantitative immunohistochemistry assay, a radioimmunoassay, an immunofluorescence assay, a flow cytometry assay, a protein microarray assay, an electrochemiluminescence assay, a microbead assay, a microfluidic immunoassay, or a surface plasmon resonance immunoassay.

In a specific example, the quantitative immunoassay is an ELISA that employs a polyclonal rabbit sera that contains antibodies specifically binding to FDP. In another example, the quantitative immunoassay is an ELISA that employs monoclonal antibodies specifically binding to FDP. The ELISA can be a sandwich ELISA that employs a second anti-FDP antibody to increase sensitivity.

Lastly, in the second method, the non-human blood sample is from a mammal, e.g., a dog, a cat, a rabbit, a guinea pig, a ferret, or a pig.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever.

### EXAMPLES

### Example 1: Preparation of Blood Samples

Blood samples were obtained from 262 dogs having cancer confirmed by cytology and/or histopathology. Blood samples were also obtained from 54 healthy animals. In all cases, informed consent to use clinical data for research purposes was obtained from pet owners.

Whole blood was collected in anti-coagulant-treated, i.e. ethylenediamine tetraacetic acid, tubes. The tubes were centrifuged at 1500 RPM for 10 min. immediately after collection and then the supernatant was removed and centrifuged at 16,000×g for 10 min. The resulting supernatants were diluted 1:200 in PBS for use in immunodetection assays.

### Example 2: Enzyme-Linked Immunosorbent Assay (“ELISA”)

A rabbit polyclonal serum was raised against DR-70 antigen, an antigen which is a combination of fibrin and FDP. The FDP in blood samples was quantitatively measured using the anti-DR-70 antigen antibodies in a sandwich ELISA assay as described below.

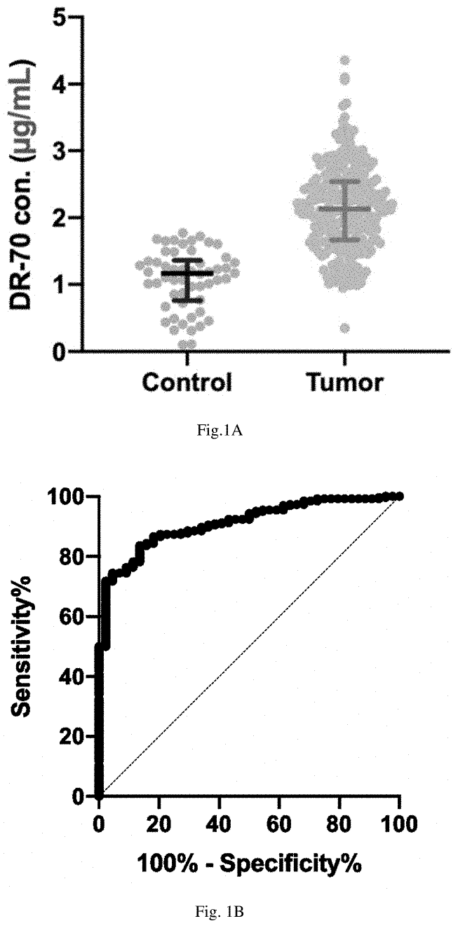

Affinity-purified anti-DR-70 rabbit serum was coated onto the wells of a 96-well plate and then incubated with 100 μL of diluted blood samples for 30 min. at room temperature. After washing the wells, a horseradish peroxidase—(“HRP”) conjugated anti-DR70 antibody was added to each well, incubated for 30 min., and washed. Detection of bound HRP-conjugated anti-DR70 antibodies was accomplished by adding tetramethylbenzidine substrate and stopping the reaction with 0.1 N HCl after sufficient color had developed. The color intensities were measured in a microplate reader at 450 nm. DR-70 concentrations were calculated by comparison to a standard curve obtained from purified DR-70. The results are show in FIG. 1A.

The median DR-70 concentration in blood samples from healthy animals was 1.163 μg/mL (0.1010-1.775 μg/ml; n=54). The median DR-70 concentration in blood samples from dogs suffering from cancer was 2.132 μg/mL (0.3495-4.355 μg/ml; n=262), a value significantly higher (p<0.0001; Kruskal-Wallis test) than that from healthy animals.

### Example 3: Diagnostic Utility

The utility of using DR-70 levels to diagnose cancer in dogs was tested by preparing a Receiver Operator Characteristics curve and calculating the area under the curve (“AUC”). The results are shown in FIG. 1B. The AUC of DR-70 was 0.9185 (95% confidence interval 0.8876 to 0.9494; p<0.0001), indicating that DR-70 levels can be used to readily discriminate between dogs with cancer and healthy animals.

The optimal cutoff value for DR-70 blood concentration was determined by calculating sensitivity and specificity at different cutoff values. The data is shown in Table 1 below.

TABLE 1
Sensitivity and Specificity of DR-70 at selected cutoff values

| | Sensi- | | Speci- | |
|---|---|---|---|---|
| DR-70 | tivity | | ficity | |
| (μg/mL) | (%) | 95% CI | (%) | 95% CI |
| >1.492 | 85.50 | 80.72% to 89.25% | 79.63 | 67.10% to 88.23% |
| >1.494 | 85.11 | 80.30% to 88.92% | 79.63 | 67.10% to 88.23% |
| >1.497 | 84.73 | 79.88% to 88.58% | 79.63 | 67.10% to 88.23% |
| >1.503 | 84.73 | 79.88% to 88.58% | 81.48 | 69.16% to 89.62% |
| >1.508 | 84.35 | 79.46% to 88.25% | 81.48 | 69.16% to 89.62% |
| >1.513 | 84.35 | 79.46% to 88.25% | 83.33 | 71.26% to 90.98% |
| >1.518 | 83.97 | 79.04% to 87.92% | 83.33 | 71.26% to 90.98% |
| >1.523 | 83.59 | 78.62% to 87.58% | 83.33 | 71.26% to 90.98% |
| >1.528 | 83.21 | 78.21% to 87.25% | 83.33 | 71.26% to 90.98% |
| >1.532 | 82.82 | 77.79% to 86.91% | 83.33 | 71.26% to 90.98% |
| >1.536 | 82.44 | 77.38% to 86.57% | 83.33 | 71.26% to 90.98% |

The optimal cutoff concentration of DR-70 was 1.513 μg/mL, having a sensitivity of 84.35% (95% CI: 79.46-88.25%) and a specificity of 83.33% (95% CI: 71.26-90.98%)

### Other Embodiments

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for quantifying fibrin/fibrinogen degradation products (FDP) in a blood sample, the method comprising:
obtaining a blood sample from a non-human animal;
centrifuging the blood sample at 10×g to 2,000×g to obtain a first supernatant;
collecting the first supernatant;
centrifuging the first supernatant at 1,000×g to 50,000×g to obtain a second supernatant;
collecting the second supernatant;
diluting the second supernatant 1:5 to 1:10,000;
contacting the diluted second supernatant with a reagent that contains antibodies specifically binding to FDP;
removing antibodies not bound to FDP; and
detecting an amount of antibodies specifically bound to FDP, thereby quantifying FDP in the blood sample.

2. The method of claim 1, wherein the blood sample is anti-coagulated.

3. The method of claim 1, wherein the non-human animal is a dog, a cat, a rabbit, a guinea pig, a ferret, or a pig.

4. The method of claim 1, wherein the anti-coagulated blood sample is centrifuged for 30 s to 30 min.

5. The method of claim 4, wherein the second supernatant is centrifuged for 30 s to 30 min.

6. The method of claim 5, wherein the second supernatant is diluted 1:50 to 1:5,000 before the contacting step.

7. The method of claim 6, wherein the second supernatant is diluted 1:100 to 1:1,000 before the contacting step.

8. A method for quantifying fibrin/fibrinogen degradation products (FDP) in a blood sample, the method comprising:
obtaining a blood sample from a non-human animal;
centrifuging the blood sample at 10×g to 2,000×g to obtain a first supernatant;
collecting the first supernatant;
centrifuging the first supernatant at 1,000×g to 50,000×g to obtain a second supernatant;
collecting the second supernatant;
diluting the second supernatant 1:5 to 1:10,000;
subjecting the diluted second supernatant to a quantitative immunoassay that specifically detects FDP, thereby quantifying the amount of FDP in the blood sample.

9. The method of claim 8, wherein the blood sample is anti-coagulated.

10. The method of claim 8, wherein the quantitative immunoassay is an enzyme-linked immunosorbent assay (ELISA), immunohistochemistry, immunofluorescence assay, flow cytometry assay, or microfluidic immunoassay.

11. The method of claim 10, wherein the quantitative immunoassay is an ELISA that employs a polyclonal rabbit sera that contains antibodies specifically binding to FDP.

12. The method of claim 10, wherein the quantitative immunoassay is an ELISA that employs monoclonal antibodies specifically binding to FDP.

13. The method of claim 9, wherein the non-human animal is a dog, a cat, a rabbit, a guinea pig, a ferret, or a pig.

14. The method of claim 9, wherein the anti-coagulated blood sample is centrifuged for 30 s to 30 min.

15. The method of claim 14, wherein the second supernatant is centrifuged for 30 s to 30 min.

16. The method of claim 15, wherein the second supernatant is diluted 1:50 to 1:5000 before the subjecting step.

17. The method of claim 16, wherein the second supernatant is diluted 1:100 to 1:1000 before the subjecting step.
